(19) United States
(12) Patent Application Publication
(10) Pub. No.: US 2020/0018306 A1
(43) Pub. Date: Jan. 16, 2020

(54) Sensor for Peristaltic Pump and Associated Methods

(71) Applicant: St. Jude Medical, Cardiology Division, Inc., St. Paul, MN (US)

(72) Inventors: Coleman Leach, Minneapolis, MN (US); Michael Kloosterboer, Minneapolis, MN (US); Brian Schmidt, Cottage Grove, MN (US); Lawrence Swanson, White Bear Lake, MN (US)

(21) Appl. No.: 16/506,665

(22) Filed: Jul. 9, 2019

Related U.S. Application Data
(60) Provisional application No. 62/697,191, filed on Jul. 12, 2018.

(51) Int. Cl. F04B 51/00; G01K 7/02; G01K 13/00; G05D 7/06; G01F 1/34; F04B 43/12; F04B 49/00; A61M 3/02

(52) U.S. Cl. CPC F04B 51/00; G01K 7/02; G01K 13/00; G05D 7/0676; G01F 1/34; F04B 43/1223; A61M 2205/3368; A61M 3/0254; F04B 2205/05; F04B 2205/09; F04B 2205/11; A61B 2218/002; A61M 2205/3331; F04B 49/00

(57) ABSTRACT

A sensor used to control a peristaltic pump includes a housing, a tubing channel extending through the housing, a pressure sensor adjacent the tubing channel to measure an internal pressure of a tubing set inserted into the tubing channel, and a temperature sensor adjacent the tubing channel to measure a temperature of a wall of the tubing set. The sensor can also include a bubble sensor adjacent the tubing channel to detect bubbles within a fluid flowing through the tubing set. The sensor can be used to control a peristaltic pump. For instance, the peristaltic pump can include a microprocessor that can determine the internal pressure of the tubing set from an output of the pressure sensor and compute therefrom a pump factor required to maintain a given flow rate of the fluid flowing through the tubing set. It can then adjust a pump factor of the pump head to the computed pump factor.

### CROSS-REFERENCE TO RELATED APPLICATIONS

This application claims the benefit of U.S. provisional application No. 62/697,191, filed 12 Jul. 2018, which is hereby incorporated by reference as though fully set forth herein.

### BACKGROUND

The present disclosure relates generally to irrigation pumps. In particular, the present disclosure relates to an integrated, multi-purpose sensor for use in peristaltic pumps.

Catheters are used for an ever-growing number of medical procedures. To name just a few examples, catheters are used for diagnostic, therapeutic, and ablative procedures. Typically, the physician manipulates the catheter through the patient's vasculature to the intended site, such as a site within the patient's heart. The catheter typically carries one or more electrodes (in the case of so-called “electrophysiology catheters”) or other diagnostic or therapeutic devices, which can be used for ablation, diagnosis, cardiac mapping, or the like.

Irrigated electrophysiology catheters are also known. An irrigated electrophysiology catheter is an electrophysiology catheter that is equipped to deliver an irrigation fluid, such as saline, to a location proximate the electrodes. The irrigation fluid serves, for example, to cool the electrodes or to disperse body fluids therefrom, to cool or bathe surrounding tissue, and/or to couple the electrodes to the tissue surface in the case of relatively highly conductive fluid(s).

In many irrigated electrophysiology catheters, a peristaltic pump is used to deliver the irrigation fluid. Typical peristaltic pumps operate by rotating a number of rollers mounted on a rotor to periodically compress an irrigation tube between the rollers and a pump housing or clamp, which forces the irrigation fluid through the irrigation tube.

It is desirable for a peristaltic pump to deliver a reliable and consistent flow rate of an irrigation fluid when in use. Environmental factors, such as temperature (both ambient temperature and the temperature of the irrigation fluid) and tubing set internal pressure, however, can impact the flow rate of the irrigation fluid and can necessitate adjustment of the rate at which the pump head turns in order to ensure a substantially constant flow of irrigation fluid.

### BRIEF SUMMARY

Disclosed herein is a sensor for use in controlling a peristaltic pump. The sensor includes: a housing; a tubing channel extending through the housing; a pressure sensor adjacent the tubing channel to measure an internal pressure of a tubing set inserted into the tubing channel; and a temperature sensor adjacent the tubing channel to measure a temperature of a wall of the tubing set.

In embodiments of the disclosure, the housing includes: a body that defines the tubing channel; a door including a rib; and a hinge connecting the door to the body, wherein, when the door is closed over the body, the rib extends into the tubing channel, and wherein an extent to which the rib extends into the tubing channel is selected to minimize door lift when the fluid is flowing through the tubing set.

A height of the pressure sensor into the tubing channel can be selected to prevent exceeding a calibrated operating range of the pressure sensor.

In aspects of the disclosure, the temperature sensor includes a thermocouple.

It is also contemplated that the sensor can include a membrane between the pressure sensor and the tubing channel.

In aspects of the disclosure, the pressure sensor includes a load button and a strain gauge.

Also disclosed herein is a peristaltic pump, including: a pump body including a pump head; and a sensor positioned adjacent the pump head. The sensor includes: a housing; a tubing channel extending through the housing; a pressure sensor adjacent the tubing channel to measure an internal pressure of a tubing set inserted into the tubing channel; and a temperature sensor adjacent the tubing channel to measure a temperature of a wall of the tubing set. The sensor can also include a bubble sensor adjacent the tubing channel to detect bubbles within a fluid flowing through the tubing set.

In embodiments disclosed herein, the sensor is positioned on an outlet side of the pump head.

It is also contemplated that the peristaltic pump can include: a microprocessor; and a non-transitory computer-readable medium that stores therein a program that causes the microprocessor to execute a process including: determining the internal pressure of the tubing set from an output of the pressure sensor; and computing a pump factor required to maintain a given flow rate of the fluid flowing through the tubing set based on the determined internal pressure of the tubing set; and adjusting a pump factor of the pump head to the computed pump factor. The process can further include receiving the temperature of the wall of the tubing set from the temperature sensor, and the computing step can further include computing a pump factor required to maintain a given flow rate of the fluid flowing through the tubing set based on the determined internal pressure of the tubing set and the received temperature of the wall of the tubing set.

According to another aspect of the disclosure, the computing step can further include computing a pump factor required to maintain a given flow rate of the fluid flowing through the tube based on the determined internal pressure of the tubing set and at least one property of the tubing set, such as an inner diameter of the tubing set, an outer diameter of the tubing set, and a durometer of the tubing set.

The housing of the first sensor can include: a body that defines the tubing channel; a door including a rib; and a hinge connecting the door to the body, wherein, when the door is closed over the body, the rib extends into the tubing channel, and wherein an extent to which the rib extends into the tubing channel is selected to minimize door lift when the fluid is flowing through the tubing set. A height of the pressure sensor into the tubing channel can also be selected to prevent exceeding a calibrated operating range of the pressure sensor.

In embodiments disclosed herein, the peristaltic pump further includes a membrane between the pressure sensor and the tubing channel.

The instant disclosure also provides a method of operating a peristaltic pump including a pump head, including the steps of: measuring, using a sensor, an internal pressure of a tubing set passing through the peristaltic pump; computing a pump factor required to maintain a given flow rate of the fluid flowing through the tubing set based on the measured internal pressure of the tubing set; and adjusting a pump factor of the pump head to the computed pump factor. The sensor includes: a housing; a tubing channel extending through the housing to receive the tubing set; a pressure sensor adjacent the tubing channel to measure an internal pressure of the tubing set; and a temperature sensor adjacent the tubing channel to measure a temperature of a wall of the tubing set.

The method can also include measuring, using the sensor, the temperature of the wall of the tubing set, and the computing step can include computing the pump factor required to maintain the given flow rate of the fluid flowing through the tubing set based on the measured internal pressure of the tubing set and the measured temperature of the wall of the tubing set.

In other aspects of the disclosure, the computing step can include computing the pump factor required to maintain the given flow rate of the fluid flowing through the tubing set based on the measured internal pressure of the tubing set and at least one property of the tubing set.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 depicts an exemplary peristaltic pump.

FIG. 2 is an exploded view of the exemplary peristaltic pump of FIG. 1.

FIG. 3 is a perspective view of a first sensor according to embodiments of the disclosure.

FIG. 4 is a cross-section of the first sensor of FIG. 3 taken along line 4-4 in FIG. 3.

FIG. 5 is a cross-section of the first sensor of FIG. 3 taken along line 5-5 in FIG. 3.

FIG. 6 is a representative plot of analog-to-digital converter (ADC) count versus time for a tubing set at atmospheric pressure within the first sensor of FIG. 3.

FIG. 7 is a representative plot of temperature factor versus temperature measured by the first sensor of FIG. 3.

FIG. 8 is a representative plot of internal pressure of a tubing set versus net ADC count output by the first sensor of FIG. 3.

FIG. 9 is an illustrative lookup table corresponding to the representative plot of FIG. 7.

FIG. 10 is an illustrative lookup table corresponding to the representative plot of FIG. 8.

FIG. 11 is a representative plot that relates pump factor to flow rate for various operating pressure ranges at a given operating temperature range.

FIG. 12 is an illustrative lookup table corresponding to the representative plot of FIG. 11.

While multiple embodiments are disclosed, still other embodiments of the present disclosure will become apparent to those skilled in the art from the following detailed description, which shows and describes illustrative embodiments. Accordingly, the drawings and detailed description are to be regarded as illustrative in nature and not restrictive.

### DETAILED DESCRIPTION

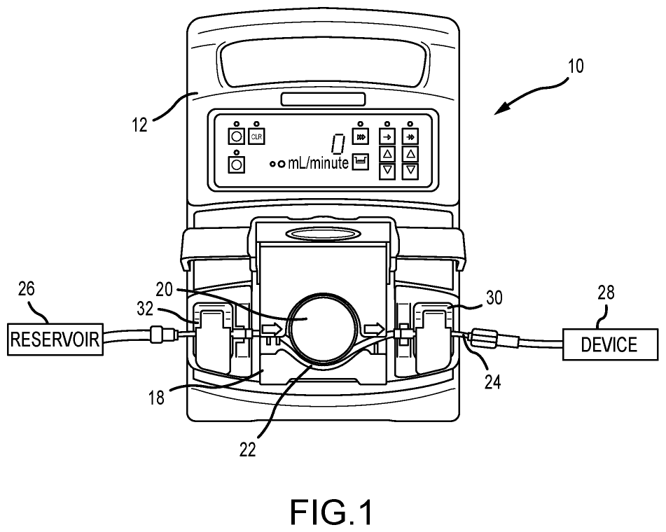

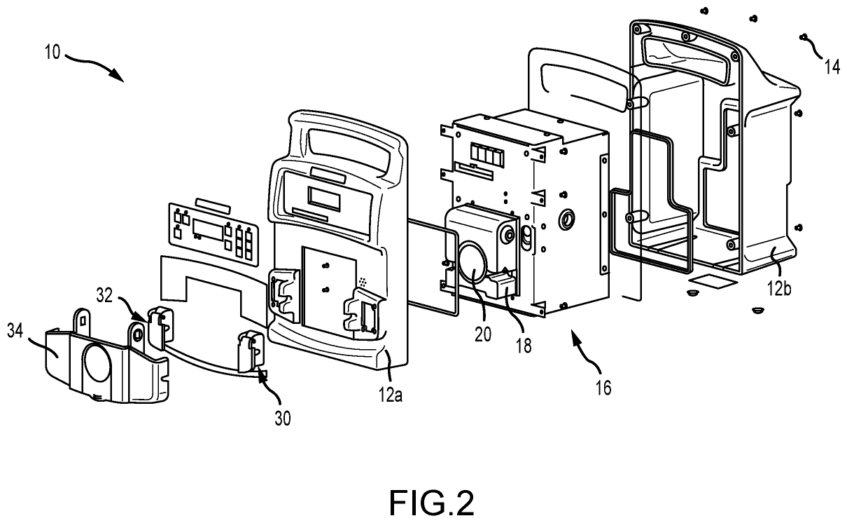

Referring now to the figures, FIGS. 1 and 2 show a peristaltic pump 10. The basic configuration and operation of peristaltic pump 10 will be familiar to those of ordinary skill in the art, such that a detailed explanation thereof is not necessary herein. Instead, the instant disclosure will focus on those features of peristaltic pump 10 pertinent to an understanding of the instant teachings.

Peristaltic pump 10 includes an enclosure 12. As shown in the exploded view of FIG. 2, enclosure 12 includes a front piece 12a and a rear piece 12b, which can be secured to each other using a plurality of fasteners (e.g., screws) 14.

Enclosure 12 encompasses a pump body 16, which in turn includes a pump clamp 18 and a pump head 20 that can rotate relative to pump body 16. A channel 22, which accommodates a tubing set 24, is defined between pump clamp 18 and pump head 20. One end of tubing set 24 can be coupled to a suitable reservoir of irrigation fluid 26 (shown schematically), while the opposite end of tubing set 24 can be coupled to a medical device 28, such as an irrigated electrophysiology catheter (shown schematically).

Peristaltic pump 10 also includes a first sensor 30 and a second sensor 32, which are positioned on opposite sides of pump head 20. More particularly, first sensor 30 can be positioned on an outlet side of pump head 20, while second sensor 32 can be positioned on an inlet side of pump head 20. First and second sensors 30, 32 are discussed in further detail below.

In embodiments, peristaltic pump 10 can also include a door 34. Door 34 covers, inter alia, pump clamp 18, tubing set 24, and first and second sensors 30, 32.

As those of ordinary skill in the art will recognize, when peristaltic pump 10 is in operation, pump head 20 turns, causing a series of rollers on the circumference of pump head 20 to sequentially impinge on tubing set 24 and push it against pump clamp 18. This forces fluid through tubing set 24 and provides a flow of irrigation fluid from reservoir 26 to medical device 28. The rate at which pump head 20 rotates is referred to herein as the “pump factor,” while the rate at which fluid is delivered to medical device 28 is referred to herein as the “flow rate.”

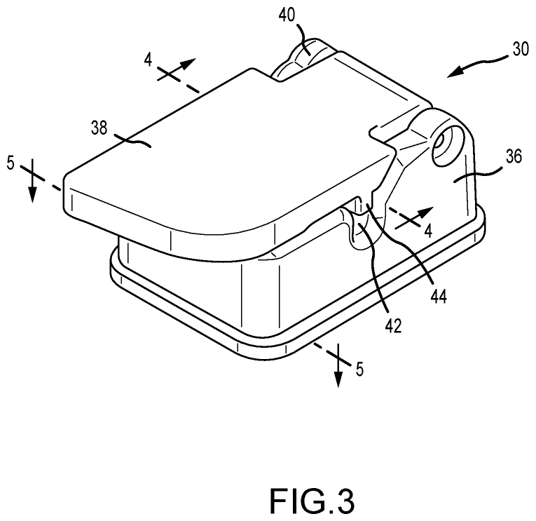

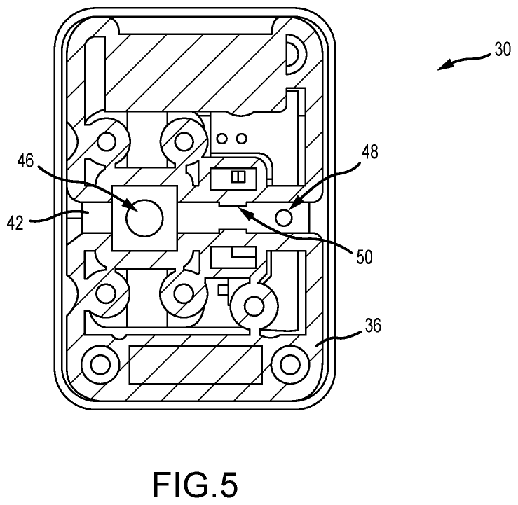

FIGS. 3-5 depict first sensor 30. As seen in FIG. 3, first sensor 30 generally includes a housing made up of a body 36 and a door 38 attached to body 36 by a hinge 40.

Body 36 defines a tubing channel 42 that extends through the housing. As described in further detail below, tubing channel 42 accommodates tubing set 24 when peristaltic pump 10 is in use. To facilitate retention of tubing set 24 within tubing channel 42, it is desirable for door 38 to be positioned flush against body 36 when the former is closed over the latter. The housing can also incorporate a magnetic closure to secure door 38 to body 36 when the former is closed over the latter.

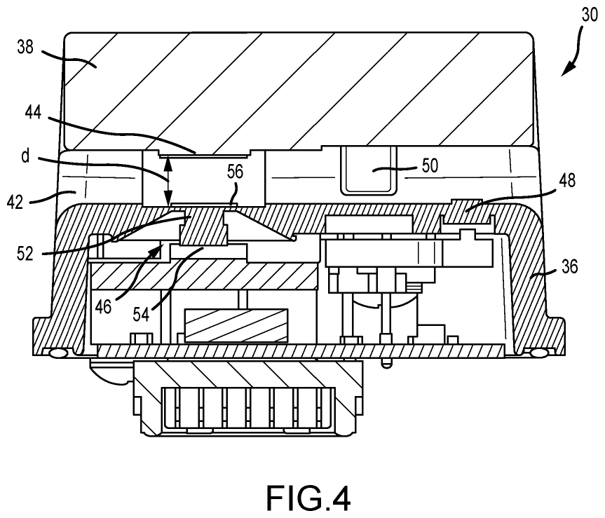

Door 38 includes a rib 44. When door 38 is closed over body 36 (e.g., as shown in FIGS. 3 and 4), rib 44 extends into tubing channel 42, and impinges on tubing set 24 when inserted into tubing channel 42. It follows that, as fluid flows through tubing set 24 when peristaltic pump 10 is in use, tubing set 24 will push against rib 44. It is therefore desirable to select the height of rib 44 above door 38 to minimize the likelihood that door 38 will lift away from body 36 when peristaltic pump 10 is in use, in particular when the fluid flowing through tubing set 24 is at high pressure.

First sensor 30 integrates a plurality of sensors into its housing (and, more particularly, into body 36). In embodiments of the disclosure, these sensors can include a pressure sensor 46, a temperature sensor 48, and a bubble sensor 50. Advantageously, incorporating pressure sensor 46, temperature sensor 48, and bubble sensor 50 into a single device helps reduce the cost of tubing set 24 by eliminating sensors from tubing set 24.

Pressure sensor 46 is positioned adjacent tubing channel 42 in order to measure the internal pressure of tubing set 24. Those of ordinary skill in the art will, from reading this disclosure, understand and appreciate how to select an appropriate pressure sensor 46 for a given use of first sensor 30. In one exemplary embodiment of first sensor 30, however, pressure sensor 46 includes a load button 52 and a strain gauge 54. Load button 52 extends into tubing channel 42 and is in contact with the wall of tubing set 24. Consequently, changes in the internal pressure of tubing set 24 displace load button 52, which in turn changes the output signal of strain gauge 54.

It should be understood, however, that the height of pressure sensor 46 into tubing channel 42 should be selected to ensure that pressure sensor 46 does not exceed its calibrated operating range during use of peristaltic pump 10. For instance, the extent to which load button 52 protrudes into tubing channel 42 should be selected to ensure that the anticipated operating pressures within tubing set 24 will not exceed the measurement capacity of strain gauge 54.

Furthermore, from the foregoing disclosure, one of ordinary skill in the art will also understand that selecting the extent to which load button 52 protrudes into tubing channel 42 should also take into account the height of rib 44 on door 38. In other words, design of first sensor 30 considers the depth d of tubing channel 42 between rib 44, on one side, and load button 52, on the other side. In embodiments of the disclosure, d is less than the outer diameter of tubing set 24, such that tubing set 24 is compressed by between about 2% and about 4% when door 38 is closed. For instance, for a tubing set 24 having an outer diameter of about 0.125 inches, d can be about 0.121 inches, resulting in about 3.2% compression.

Embodiments of first sensor 30 can include a membrane 56 between pressure sensor 46 and tubing set 24. One suitable material for membrane 56 is COHRlastic® 9235 solid silicone rubber from Saint-Gobain North America of Malvern, Pa.

Temperature sensor 48 is also positioned adjacent tubing channel 42, allowing it to measure the temperature of the wall of tubing set 24. Those of ordinary skill in the art will, from reading this disclosure, understand and appreciate how to select an appropriate temperature sensor 48 for a given use of first sensor 30. In one exemplary embodiment of first sensor 30, however, temperature sensor 48 is a thermocouple.

As with other aspects of first sensor 30 described above, the extent to which temperature sensor 48 extends into tubing channel 42 can be selected to minimize door lift during operation of peristaltic pump 10.

Bubble sensor 50 is also positioned adjacent tubing channel 42, which allows it to detect bubbles within a fluid flowing through tubing set 24 when peristaltic pump 10 is in use. Those of ordinary skill in the art will, from reading this disclosure, understand and appreciate how to select an appropriate bubble sensor 50 for a given use of first sensor 30 (based on, for example, the nominal diameter of tubing set 24 and the bubble size to be detected). Suitable sensors can be purchased commercially, for example, from Strain Measurement Devices Inc. of Wallingford, Conn.

The construction of second sensor 32 can be similar to the construction of first sensor 30 (e.g., second sensor 32 can also a housing, made up of a door and a body, and that defines therethrough a tubing channel to accommodate tubing set 24). In embodiments of the disclosure, however, second sensor 32 includes only a bubble sensor.

As those of ordinary skill in the art will recognize, the flow rate of a peristaltic pump is a function of several variables, including, without limitation, the inner diameter, outer diameter, and durometer of the tubing set (e.g., tubing set 24), the pressure of the fluid being pumped, the temperature of the fluid being pumped, and the pump factor. Peristaltic pump 10 according to embodiments disclosed herein thus includes a variable pump factor, which allows it to maintain a desired flow rate despite changes in other variables that might otherwise impact flow rate (e.g., increases and/or decreases in operating temperature and/or pressure).

As discussed above, hardware characteristics can impact flow rate. For instance, changes in tubing set 24 (e.g., using a tubing set with different inner and/or outer diameters, a different durometer, and/or a different key spacing that changes the stretch of the tubing set when installed into the pump; manufacturing variations in tubing set 24; and/or variations in the properties of tubing set 24 introduced during sterilization and packaging or through use of tubing set 24, etc.) and/or to peristaltic pump 10 (e.g., altering the dimensions of channel 22, the diameter of pump head 20, the diameter of the rollers on pump head 20, and/or the number of rollers on pump head 20, etc.) can result in changes to the pump factor required to achieve a particular flow rate at a particular operating temperature and/or operating pressure. It should therefore be understood that a variable pump factor determined according to the teachings herein is specific to a particular peristaltic pump, including its sensors, and a particular tubing set. For instance, the exemplary variable pump factor described herein relates to a representative tubing set 24 made of Tygon® ND-100-65 tubing from St. Gobain North America that has an inner diameter of about 1/16 inch, an outer diameter of about ⅛ inch proximate first sensor 30, and an outer diameter of about 3/16 inch proximate pump head 20.

Accordingly, the following description of the development of an exemplary variable pump factor should be regarded as illustrative, not limiting. Indeed, those of ordinary skill in the art will appreciate how to apply the teachings herein to the development of variable pump factors more generally (e.g., one of ordinary skill in the art could apply the teachings herein to develop a variable pump factor that relates pump factor, flow rate, temperature, and pressure for any given hardware).

In embodiments of the disclosure, the variable pump factor can be implemented in the firmware of peristaltic pump 10. In other words, the variable pump factor disclosed herein can be embodied as a computer program stored in a non-transitory computer-readable medium for execution on one or more microprocessors. Alternatively, the variable pump factor disclosed herein can be hardware implemented (e.g., as a collection of logic gates).

As discussed above, a variable pump factor according to aspects of the disclosure utilizes experimentally-derived data to determine the pump factor necessary to achieve a given flow rate for a particular operating temperature and/or operating pressure. One experimentally-derived quantity utilized in aspects of the disclosure is the baseline force exerted by tubing set 24 with tubing set 24 at atmospheric pressure (e.g., no internal tubing pressure). This quantity is referred to herein as the “initial force offset.”

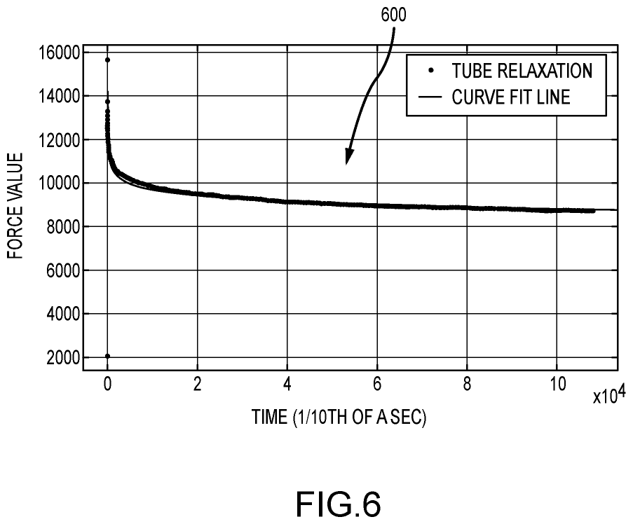

Specifically, when tubing set 24 is inserted into tubing channel 42 and door 38 is closed, there is a spike in analog-to-digital converter (“ADC”) counts from pressure sensor 46 (because of the force that rib 44 exerts on the wall of tubing set 24). As tubing set 24 relaxes, however, ADC counts from pressure sensor 46 decay. The decayed level defines the initial force offset. FIG. 6 illustrates a curve 600 of ADC counts versus time for the representative tubing set 24; the initial force offset defined by curve 600 is about 10000 counts.

With the initial force offset determined, pressure sensor 46 can measure the internal pressure P of tubing set 24. In aspects of the disclosure, the internal pressure P can be defined as

$$P = {D ( \frac{ADC}{TF} )}^{3} + {E ( \frac{ADC}{TF} )}^{2} + F ( \frac{ADC}{TF} ) ,$$

where ADC is the difference between the instantaneous ADC count from pressure sensor 46 and the initial force offset (referred to herein as the “net ADC count”), TF is a temperature factor as discussed below, and D, E, and F are experimentally-determined coefficients.

The temperature factor TF relates the stiffness of tubing set 24 to temperature (e.g., as temperature increase, tubing set 24 becomes more pliable and vice versa), and in turn relates variations in the stiffness of tubing set 24 to the output of pressure sensor 46 (e.g., at constant internal pressure, tubing set 24 will exert less force on load button 52, and pressure sensor 46 will output fewer ADC counts, as temperature increases). The temperature factor TF is therefore a function of the temperature measured by temperature sensor 48. More particularly, the temperature factor TF can be defined as TF=AT²+BT+C, where T is the temperature measured by temperature sensor 48 and A, B, and C are experimentally-determined coefficients.

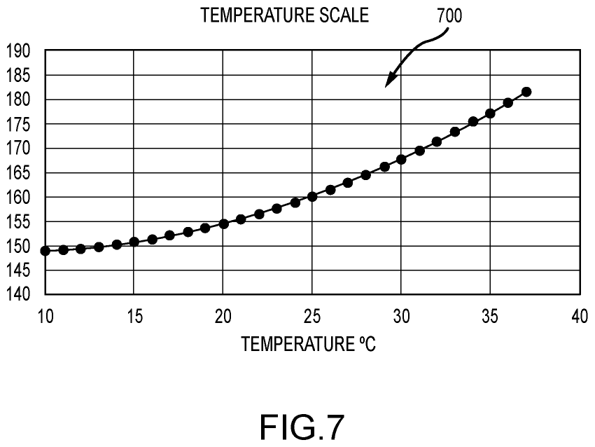

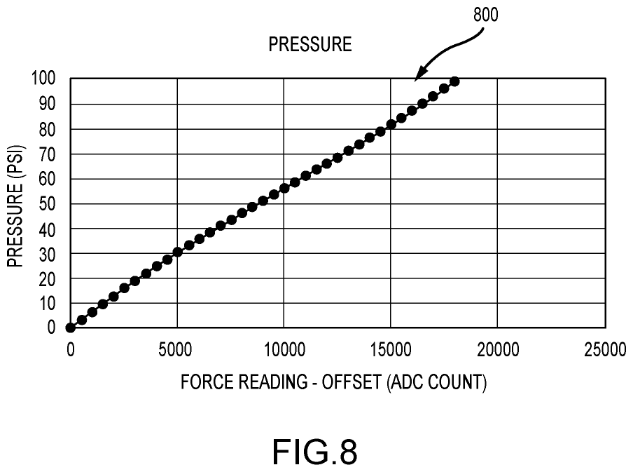

By generating a data set that includes pressure and temperature information for a peristaltic pump 10, first sensor 30, and tubing set 24 in operation, one can derive coefficients A, B, C, D, E, and F, for example via well-understood curve-fitting techniques. FIGS. 7 and 8 respectively illustrate a first curve 700 that relates temperature factor TF to temperature as measured by temperature sensor 48 and a second curve 800 that relates the pressure within tubing set 24 to the net ADC count of pressure sensor 46.

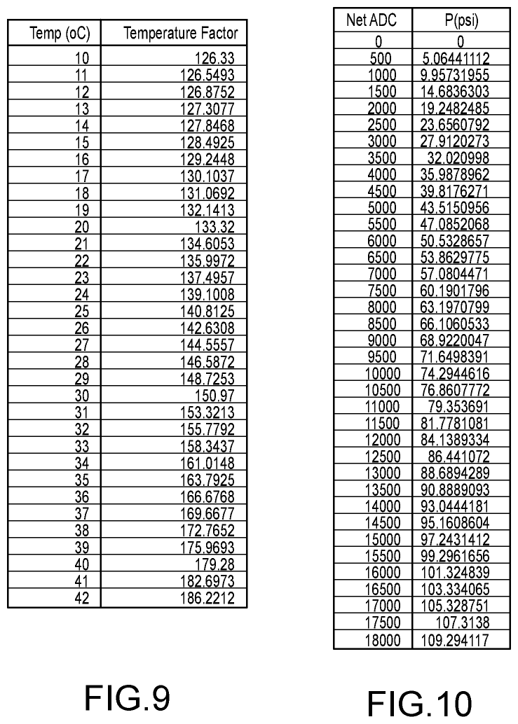

The results (that is, the experimentally-determined pressure-to-net ADC count and temperature factor to temperature relationships) can be implemented in peristaltic pump 10 (e.g., embedded in firmware) as equations, curves, and/or lookup tables. For the sake of illustration, FIG. 9 is an exemplary temperature factor lookup table that corresponds to curve 700 of FIG. 7, while FIG. 10 is an exemplary pressure lookup table that corresponds to curve 800 of FIG. 8.

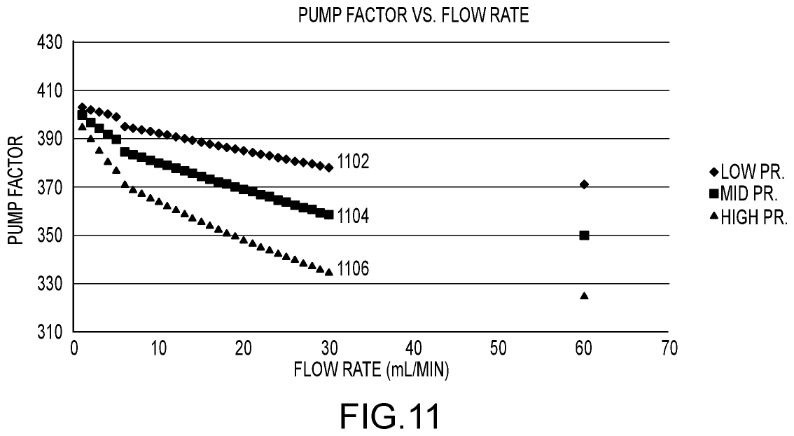

In turn, the foregoing relationships allow experimental determination of the relationship between pump factor and flow rate at any operating temperature and pressure values or ranges. For example, FIG. 11 shows several plots relating pump factor to flow rate (in mL/min) for data collected at operating temperatures between about 15 degrees and about 35 degrees Celsius. The data of plot 1102 was collected at about atmospheric pressure; the data of plot 1104 was collected at operating pressures between about 20 psi and about 40 psi above atmospheric pressure; and the data of plot 1106 was collected at operating pressures between about 40 psi and about 85 psi above atmospheric pressure.

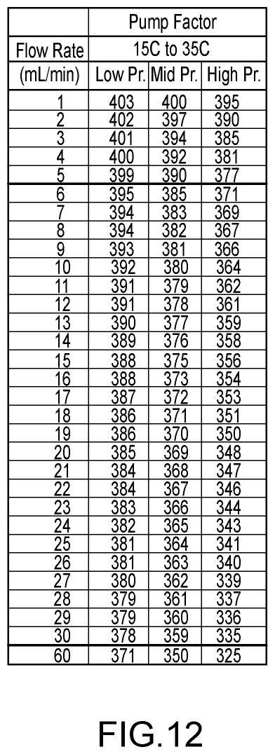

A curve, such as a second-order polynomial curve, can be fit to each plot 1102, 1104, 1106 in order to develop a corresponding lookup table, such as illustrated in FIG. 12. The variable pump factor can be implemented in peristaltic pump 10, e.g., by embedding the equations, curves, and/or lookup table into its firmware.

The variable pump factor described above can be leveraged during use of peristaltic pump 10. Using the outputs of pressure sensor 46 and/or temperature sensor 48, peristaltic pump 10 (e.g., a microprocessor therein) can determine the instantaneous operating temperature and pressure (e.g., using curves 700, 800 and/or their corresponding lookup tables as stored in the firmware of peristaltic pump 10). From the instantaneous operating temperature and pressure, peristaltic pump 10 (e.g., a microprocessor therein) can compute the appropriate pump factor for a given (e.g., user set) flow rate by utilizing the applicable variable pump factor equations, curves, and/or lookup tables as described above (e.g., plots 1102, 1104, 1106 and/or the lookup table of FIG. 12 as stored in the firmware of peristaltic pump 10) and adjust the pump factor of pump head 20 accordingly.

Although several embodiments have been described above with a certain degree of particularity, those skilled in the art could make numerous alterations to the disclosed embodiments without departing from the spirit or scope of this invention.

For example, although peristaltic pump 10 is described as having a variable pump factor implemented with a single temperature range and three pressure ranges, those of ordinary skill in the art will appreciate how to extend the teachings herein to any number of temperature and/or pressure ranges.

Likewise, although peristaltic pump 10 is described above as having a single variable pump factor for a single tubing set, it is contemplated that peristaltic pump 10 can instead include multiple variable pump factors, each one corresponding to one of a plurality of tubing sets having differing inner diameters, outer diameters, durometers, and/or the like. Depending upon the specific tubing set utilized in a particular procedure, the appropriate variable pump factor can be selected (e.g., from multiple variable pump factors stored in the firmware of peristaltic pump 10). Selection of the appropriate variable pump factor can be based on a manual input (e.g., the practitioner utilizes an interface on peristaltic pump 10 to select the tubing set in use in the procedure from a catalog) and/or automatic (e.g., peristaltic pump 10 reads the tubing set in use and selects the corresponding variable pump factor).

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A sensor for use in controlling a peristaltic pump, the sensor comprising:
a housing;
a tubing channel extending through the housing;
a pressure sensor adjacent the tubing channel to measure an internal pressure of a tubing set inserted into the tubing channel; and
a temperature sensor adjacent the tubing channel to measure a temperature of a wall of the tubing set.

2. The sensor according to claim 1, wherein the housing comprises:
a body that defines the tubing channel;
a door including a rib; and
a hinge connecting the door to the body,
wherein, when the door is closed over the body, the rib extends into the tubing channel, and
wherein an extent to which the rib extends into the tubing channel is selected to minimize door lift when the fluid is flowing through the tubing set.

3. The sensor according to claim 1, wherein a height of the pressure sensor into the tubing channel is selected to prevent exceeding a calibrated operating range of the pressure sensor.

4. The sensor according to claim 1, wherein the temperature sensor comprises a thermocouple.

5. The sensor according to claim 1, further comprising a membrane between the pressure sensor and the tubing channel.

6. The sensor according to claim 1, wherein the pressure sensor comprises a load button and a strain gauge.

7. A peristaltic pump, comprising:
a pump body including a pump head; and
a sensor positioned adjacent the pump head, the sensor comprising:
a housing;
a tubing channel extending through the housing;
a pressure sensor adjacent the tubing channel to measure an internal pressure of a tubing set inserted into the tubing channel; and
a temperature sensor adjacent the tubing channel to measure a temperature of a wall of the tubing set.

8. The peristaltic pump according to claim 7, wherein the sensor further comprises a bubble sensor adjacent the tubing channel to detect bubbles within a fluid flowing through the tubing set.

9. The peristaltic pump according to claim 7, wherein the sensor is positioned on an outlet side of the pump head.

10. The peristaltic pump according to claim 7, further comprising:
a microprocessor; and
a non-transitory computer-readable medium that stores therein a program that causes the microprocessor to execute a process comprising:
determining the internal pressure of the tubing set from an output of the pressure sensor; and
computing a pump factor required to maintain a given flow rate of the fluid flowing through the tubing set based on the determined internal pressure of the tubing set; and
adjusting a pump factor of the pump head to the computed pump factor.

11. The peristaltic pump according to claim 10, wherein:
the process further comprises receiving the temperature of the wall of the tubing set from the temperature sensor; and
the computing step further comprises computing a pump factor required to maintain a given flow rate of the fluid flowing through the tubing set based on the determined internal pressure of the tubing set and the received temperature of the wall of the tubing set.

12. The peristaltic pump according to claim 10, wherein the computing step further comprises computing a pump factor required to maintain a given flow rate of the fluid flowing through the tube based on the determined internal pressure of the tubing set and at least one property of the tubing set.

13. The peristaltic pump according to claim 12, wherein the at least one property of the tubing set comprises at least one of an inner diameter of the tubing set; an outer diameter of the tubing set; and a durometer of the tubing set.

14. The peristaltic pump according to claim 7, wherein the housing of the first sensor comprises:
a body that defines the tubing channel;
a door including a rib; and
a hinge connecting the door to the body,
wherein, when the door is closed over the body, the rib extends into the tubing channel, and
wherein an extent to which the rib extends into the tubing channel is selected to minimize door lift when the fluid is flowing through the tubing set.

15. The peristaltic pump according to claim 7, wherein a height of the pressure sensor into the tubing channel is selected to prevent exceeding a calibrated operating range of the pressure sensor.

16. The peristaltic pump according to claim 7, further comprising a membrane between the pressure sensor and the tubing channel.

17. A method of operating a peristaltic pump including a pump head, the method comprising:
measuring, using a sensor, an internal pressure of a tubing set passing through the peristaltic pump, the sensor comprising:
a housing;
a tubing channel extending through the housing to receive the tubing set;
a pressure sensor adjacent the tubing channel to measure an internal pressure of the tubing set; and
a temperature sensor adjacent the tubing channel to measure a temperature of a wall of the tubing set; and
computing a pump factor required to maintain a given flow rate of the fluid flowing through the tubing set based on the measured internal pressure of the tubing set; and
adjusting a pump factor of the pump head to the computed pump factor.

18. The method according to claim 17, further comprising:
measuring, using the sensor, the temperature of the wall of the tubing set,
wherein the computing step further comprises computing the pump factor required to maintain the given flow rate of the fluid flowing through the tubing set based on the measured internal pressure of the tubing set and the measured temperature of the wall of the tubing set.

19. The method according to claim 17, wherein the computing step further comprises computing the pump factor required to maintain the given flow rate of the fluid flowing through the tubing set based on the measured internal pressure of the tubing set and at least one property of the tubing set.
